(19) United States
(12) Patent Application Publication
(10) Pub. No.: US 2023/0293090 A1
(43) Pub. Date: Sep. 21, 2023

(54) NEUROPHYSIOLOGICAL ASSESSMENT, IDENTIFICATION, PERMISSION CONTROL, MONITORING, AND NOTIFICATION SYSTEM FOR COVID-19

(71) Applicant: Great Plain Technologies LLC, Pittston, PA (US)

(72) Inventors: Clifton R. LACY, Highland Park, NJ (US); Warren S. Gifford, Monroe Township, NJ (US); David L. Turock, Fort Lauderdale, FL (US)

(21) Appl. No.: 17/700,124

(22) Filed: Mar. 21, 2022

(51) Int. Cl. A61B 5/00; A61B 5/16; A61B 5/18; G16H 50/20

(52) U.S. Cl. CPC A61B 5/4088; A61B 5/163; A61B 5/4035; A61B 5/18; G16H 50/20

(57) ABSTRACT

Systems and methods for controlling permissions for an individual. The techniques can include presenting neurophysiological stimuli to the individual, measuring neurophysiological response by the individual to the neurophysiological stimuli, determining whether the individual is exhibiting cognitive impairment based on whether the measured neurophysiological response differs from a reference response by a threshold, and controlling a permission according to criteria and the determination as to whether the individual is exhibiting cognitive impairment, wherein the permission allows the individual to at least one of access a resource or perform an action. The systems and methods described herein address COVID-19 related issues by screening for detection of findings compatible with COVID-19, providing a mechanism to both verify the identity of the individual and to link SARS-CoV-2 laboratory test results to that individual, and controlling permission to access resources or perform actions to prevent transmission of COVID-19.

### BACKGROUND

This innovation assesses the impact of sensory, cognitive, and motor impairment caused by sleep deprivation, fatigue, aging, disease, injury, and substance intoxication on understanding, judgment, and performance. It determines whether the individual meets the criteria for identity, access, and actions, and can monitor and update determination on an ongoing basis. This is a globally important personal, public health, security, economic, and safety issue.

From a personal and public health perspective, these forms of impairment can have negative health consequences (including injury and death) and contribute to social, economic, and safety harms (for example, decreased productivity, increased insurance costs, breaches in security, and social disruption). Countless numbers of workers are in safety-sensitive and security-sensitive jobs, including manufacturing, construction, agriculture, transportation, utilities, public safety, homeland security, healthcare, and military, among others. An individual's sensory, cognitive, and/or motor impairment can confer significant risk of injury and/or death, property damage, financial losses, and security breaches.

It is critically important to identify individuals at risk for inadvertent harm to self and others before damaging incidents occur. Significant examples of this problem include impaired driving of personal or work vehicles and impaired operation of potentially dangerous industrial equipment. One major use case of this innovation is in detection and identification of impairments due to intoxication by medications, drugs, alcohol, or illness. The key determinant is not the use of the substances, but rather the presence or absence of sensory, cognitive, or motor impairment that limits or disrupts the necessary ability to perform actions relevant to the task at hand.

The impact on worker safety of medication and psychoactive substance use and abuse is well recognized; however, drug testing has very limited value in detection of impairment and prevention of harm. Breathalyzer testing has long been utilized by law enforcement to detect driving under the influence of alcohol. Employers have implemented programs of random drug testing to detect illicit drug use in the workplace. However, these tests are intrusive and unpleasant for the individual, often have delayed turnaround times for results reporting, can be expensive, and cannot determine the actual ability of the individual to perform the authorized access and actions. Blood, urine, saliva, and exhaled breath testing may be able to detect alcohol, cannabis-related THC, illicit drugs, and/or prescription medications, but may not be able to differentiate individuals who are impaired from those who are not. In particular, some individuals may be impaired with very low blood levels of alcohol, whereas others may not be impaired at higher blood levels. For example, individuals with detectable THC in blood or saliva from use of cannabis in prior days or weeks may not be currently impaired. In addition, testing for substances in blood, saliva, urine, or exhaled breath is unable to detect sensory, cognitive, and motor impairment caused by fatigue, aging, injury, or disease.

In addition to the user cases discussed above, the innovations described herein could be used to address issues associated with COVID-19. COVID-19 is caused by Severe Acute Respiratory Syndrome Coronavirus 2 (SARS-CoV-2). This virus is highly infectious and transmissible even by persons who are asymptomatic. The virus spreads from person-to person primarily through very fine droplets and aerosol particles produced when an infected person exhales, talks, vocalizes, sneezes, or coughs. Particles containing the virus can travel more than 6 feet from the source, more so if the infected individual is coughing, yelling or singing and especially indoors and in dry conditions with relative humidity below 40%.

Work, school, and other congregate settings pose a significant risk for contagion. CDC and OSHA recommend maintaining multiple layers of protection beyond vaccination, including mask wearing, distancing, and increased ventilation. One key infection control measure is keeping infected individuals out of the workplace and other congregate settings. To accomplish this, individuals who have symptoms compatible with COVID-19 should be denied access to the workplace and other congregate settings until testing has been accomplished and then isolated from others if testing confirms COVID-19 positivity. Since a significant proportion of COVID-positive individuals are asymptomatic or pre-symptomatic during portions of the contagious period, a screening mechanism for detection of findings compatible with COVID-19 is necessary to prevent spread of disease and avoid potentially life threatening complications in individuals at high risk of severe outcomes from the disease.

During the COVID-19 pandemic, individuals used false identification and false information to game the testing system as follows:

Individuals, whose employers provided paid COVID-19 sick leave, who tested negative for SARS-CoV-2 falsely claimed to have positive results for the infection in order to qualify for paid sick leave.

Individuals, whose employers did not provide paid COVID-19 sick leave, who tested positive for SARS-CoV-2 falsely claimed to have negative results for the infection in order stay on the job and receive pay.

In the former case, the falsification results in unwarranted expense for the employer. In the latter case, the falsification potentially exposes other employees and customers to an individual with COVID-19, risking contagion and the consequences of the illness.

### SUMMARY

There are provided systems and methods for granting or denying access and/or action permission, particularly access control systems that are configured to identify sensory and/or cognitive impairment in users. The systems and methods described herein can be implemented in a wide range of use cases, including preventing individuals from operating various types of devices (including vehicles) while impaired (e.g., from alcohol, drugs, fatigue, or illness), controlling access to buildings, or controlling access to private, sensitive, or secure information such as that found in computer systems or databases.

In addition to the general benefits described above, the systems and methods described herein are also useful for addressing the above COVID-19 related issues by screening for detection of findings compatible with COVID-19 and also by providing a mechanism to both verify the identity of the individual and to link SARS-CoV-2 laboratory test results to that individual.

In some embodiments, there is provided a computer-implemented method for controlling a permission for an individual by a mobile device, the mobile device comprising a display screen, a detector, and a processor, the method comprising: presenting, by the display screen, neurophysiological stimuli to the individual; measuring, by the detector, neurophysiological response by the individual to the neurophysiological stimuli; determining, by the processor, whether the individual is exhibiting cognitive impairment compatible with COVID-19 based on whether the measured neurophysiological response differs from a reference response by a threshold; and controlling, by the processor, a permission according to criteria and the determination as to whether the individual is exhibiting the cognitive impairment compatible with COVID-19, wherein the permission allows the individual to at least one of access a resource or perform an action.

In some embodiments, there is provided a computer-implemented method for controlling a permission for an individual by a mobile device, the mobile device comprising a display screen, a detector, and a processor, the method comprising: presenting, by the display screen, neurophysiological stimuli to the individual; measuring, by the detector, neurophysiological response by the individual to the neurophysiological stimuli; determining, by the processor, whether the individual is exhibiting cognitive impairment based on whether the measured neurophysiological response differs from a reference response by a threshold; and controlling, by the processor, a permission according to criteria and the determination as to whether the individual is exhibiting cognitive impairment, wherein the permission allows the individual to at least one of access a resource or perform an action.

In some embodiments of the methods, the methods further comprise monitoring, by the processor, the individual as the individual interacts with the resource according to the permission granted; determining, by the processor, whether the individual is exhibiting the cognitive impairment compatible with COVID-19 based on the monitoring; and revoking, by the processor, the permission in response to the determination that the individual is exhibiting the cognitive impairment compatible with COVID-19.

In some embodiments of the methods, the methods further comprise monitoring, by the processor, the individual as the individual interacts with the resource according to the permission granted; determining, by the processor, whether the individual is exhibiting the cognitive impairment based on the monitoring; and revoking, by the processor, the permission in response to the determination that the individual is exhibiting the cognitive impairment.

In some embodiments of the methods, the permission comprises entering, starting, or driving a motor vehicle.

In some embodiments of the methods, the permission comprises accessing a physical or virtual environment.

In some embodiments of the methods, the environment comprises a building or other physical structure.

In some embodiments of the methods, the methods further comprise notifying a third party according to the criteria and the determination as to whether the individual is exhibiting the cognitive impairment compatible with COVID-19.

In some embodiments of the methods, the methods further comprise notifying a third party according to the criteria and the determination as to whether the individual is exhibiting the cognitive impairment.

In some embodiments of the methods, the methods further comprise identifying the individual based on an adaptive biometric according to the measured neurophysiological response exhibited by the individual.

In some embodiments of the methods, the neurophysiological response is measured using at least one of an eye tracking test, a trail-making test, or an eye-hand coordination test.

In some embodiments of the methods, the reference response comprises one or more default values.

In some embodiments of the methods, the reference response comprises at least one of a characterized response or a baseline response to the neurophysiological stimuli for the individual.

In some embodiments of the methods, the reference response comprises at least one of a characterized response of a population of individuals to the neurophysiological stimuli.

In some embodiments, there is provided a system for controlling a permission for an individual, the system comprising: a mobile device comprising: a display screen, a detector, a processor, and a memory coupled to the processor, the memory storing instructions that, when executed by the processor, cause the mobile device to: present, by the display screen, neurophysiological stimuli to the individual; measure, by the detector, neurophysiological response by the individual to the neurophysiological stimuli; determine whether the individual is exhibiting cognitive impairment compatible with COVID-19 based on whether the measured neurophysiological response differs from a reference response by a threshold; and control a permission according to criteria and the determination as to whether the individual is exhibiting the cognitive impairment compatible with COVID-19, wherein the permission allows the individual to at least one of access a resource or perform an action.

In some embodiments, there is provided a system for controlling a permission for an individual, the system comprising: a mobile device comprising: a display screen, a detector, a processor, and a memory coupled to the processor, the memory storing instructions that, when executed by the processor, cause the mobile device to: present, by the display screen, neurophysiological stimuli to the individual; measure, by the detector, neurophysiological response by the individual to the neurophysiological stimuli; determine whether the individual is exhibiting cognitive impairment based on whether the measured neurophysiological response differs from a reference response by a threshold; and control a permission according to criteria and the determination as to whether the individual is exhibiting the cognitive impairment, wherein the permission allows the individual to at least one of access a resource or perform an action.

In some embodiments of the systems, the memory stores further instructions that, when executed by the processor, cause the mobile device to: monitor the individual as the individual interacts with the resource according to the permission; determine whether the individual is exhibiting the cognitive impairment compatible with COVID-19 based on the monitoring; and revoke the permission in response to the determination that the individual is exhibiting the cognitive impairment compatible with COVID-19.

In some embodiments of the systems, the memory stores further instructions that, when executed by the processor, cause the mobile device to: monitor the individual as the individual interacts with the resource according to the permission; determine whether the individual is exhibiting the cognitive impairment based on the monitoring; and revoke the permission in response to the determination that the individual is exhibiting the cognitive impairment.

In some embodiments of the systems, the permission comprises entering, starting, or driving a motor vehicle.

In some embodiments of the systems, the permission comprises accessing a physical or virtual environment.

In some embodiments of the systems, the environment comprises a building or other physical structure.

In some embodiments of the systems, the memory stores further instructions that, when executed by the processor, cause the mobile device to: notify a third party according to the criteria and the determination as to whether the individual is exhibiting the cognitive impairment compatible with COVID-19.

In some embodiments of the systems, the memory stores further instructions that, when executed by the processor, cause the mobile device to: notify a third party according to the criteria and the determination as to whether the individual is exhibiting the cognitive impairment.

In some embodiments of the systems, the memory stores further instructions that, when executed by the processor, cause the mobile device to: identify the individual based on an adaptive biometric according to the measured neurophysiological response exhibited by the individual.

In some embodiments of the systems, the neurophysiological response is measured using at least one of an eye tracking test, a trail-making test, or an eye-hand coordination test.

In some embodiments of the systems, the reference response comprises one or more default values.

In some embodiments of the systems, the reference response comprises at least one of a characterized response or a baseline response to the neurophysiological stimuli for the individual.

In some embodiments of the systems, the reference response comprises at least one of a characterized response of a population of individuals to the neurophysiological stimuli.

### FIGURES

The accompanying drawings, which are incorporated in and form a part of the specification, illustrate the embodiments of the invention and together with the written description serve to explain the principles, characteristics, and features of the invention. In the drawings:

FIG. 1 illustrates a diagram of a permission control system, in accordance with an embodiment.

FIG. 2 illustrates a schematic diagram of a first embodiment of the permission control system of FIG. 1.

FIG. 3 illustrates a schematic diagram of a second embodiment of the permission control system of FIG. 1.

FIG. 4 illustrates a schematic diagram of a third embodiment of the permission control system of FIG. 1.

FIG. 5 illustrates a diagram of another embodiment of the permission control system.

FIG. 6 illustrates a flow diagram of a process for controlling access and/or action permissions based on cognitive impairment, in accordance with an embodiment.

### DESCRIPTION

As used herein, a “subject” or a “user” refers to a human individual.

As used herein, “COVID-19” refers to the infectious disease caused by the SARS-CoV-2 virus. It should be further noted that any measurement or analysis of COVID-19 described herein includes both the direct or indirect effects thereof, as well as its sequelae. For example, if a system is determining whether a response exhibited by a subject is compatible with COVID-19, that includes the effects both directly and indirectly caused by COVID-19, as well as its sequelae.

As used herein, a “neurophysiological stimulus” (collectively, “neurophysiological stimuli”) means anything that is configured to elicit a somatic (i.e., voluntary) and/or autonomic (i.e., involuntary) response by a subject. Neurophysiological stimuli could include, for example, olfactory stimuli, visual stimuli, or sound stimuli. Neurophysiological stimuli could include both discrete stimuli (e.g., a single scent) or more complex combinations of stimuli (e.g., a trail-making test or a finger tapping test).

As used herein, an “environment” refers to both virtual environments (e.g., computing environments) and physical environments (e.g., buildings). For example, if a system is controlling permissions associated with an environment, it could manage permissions to access resources stored within a computing environment (e.g., control whether a user can access a particular file or record) or control physical access to a building (e.g., control whether a door locks or unlocks).

As used herein, a “resource” refers to both virtual resources (e.g., a computer file or a secured portion of a website that requires a login) and physical resources (e.g., a door or a file cabinet). For example, if a system is controlling permissions associated with a resource, it could control permissions to access a portion of a website via a secured login or control the ability of the subject to physically open a door.

Permission Control Methodology

Described herein are systems and techniques for controlling users' access and/or action permissions. The access and/or action permissions can be associated with devices, locations, venues, vehicles, equipment, actions, activities, or anything else that could require permissions to be able to perform. The permission control systems described herein can determine whether an individual is cognitively impaired and control his or her access and/or action permissions accordingly. In some embodiments, the permission control systems can be configured to provide stimuli (e.g., neurophysiological stimuli) to a subject and assess the subject's response thereto in order to determine whether the subject is exhibiting signs or symptoms of cognitive impairment. In some embodiments, the permission control systems can be configured to supply olfactory stimuli to the subject and measure the subject's response (e.g., pupillary) thereto, such as is described in U.S. patent application Ser. No. 17/167,728, now U.S. Pat. No. 11,083,405, titled SYSTEMS AND METHODS FOR SCREENING SUBJECTS BASED ON PUPILLARY RESPONSE TO OLFACTORY STIMULATION, filed Feb. 4, 2021, which is hereby incorporated by reference herein in its entirety. In some embodiments, the permission control systems can be configured to supply neurophysiological stimuli (e.g., audio stimuli or visual stimuli) to the subject and measure the subject's response (e.g., trail-making test accuracy) thereto, such as is described in U.S. patent application Ser. No. 17/528,417, titled SYSTEMS AND METHODS FOR SCREENING SUBJECTS FOR NEUROPATHOLOGY ASSOCIATED WITH A CONDITION UTILIZING A MOBILE DEVICE, filed Nov. 17, 2021, which is hereby incorporated by reference herein in its entirety. Further, the neurophysiological stimuli could take the form of various testing techniques that are provided to the subject, including eye tracking, trail-making, or eye-hand coordination testing techniques.

The systems and methods described herein provide a rapid, portable, accurate, objective, access-specific, function- and activity-specific neurophysiological assessment of the subject. The systems described herein could be used to comprehensively verify individuals' identities and control permissions for access to various resources and for performance of allowed activities. In some embodiments, based on the results of the identity verification and/or testing, the systems could further notify third parties regarding failed identity verifications, whether the subjects have cognitive impairments, and so on. In some embodiments, the permission control systems can: (i) detect sensory, cognitive, and motor impairment utilizing a smart device; (ii) decide whether the individual meets the criteria for authorized identity, access, and actions; (iii) permit or deny access to physical and virtual environments, use of electronic technology and/or other equipment, and/or performance of actions; (iv) notify the individual or third parties (e.g., healthcare, supervisory, or security personnel) regarding results of testing; and (v) monitor continued performance and take appropriate actions.

In some embodiments, the systems and methods described herein can be embodied, at least in part, as software executed by a handheld smart device that assesses sensory functions, concentration, memory, visuospatial function, reaction time, decision-making, accuracy, time estimation, hand-eye coordination, neuromuscular function, motor tracking, fine motor movement, balance, and the ability to perform specific knowledge-based, skill-based, and divided-attention actions.

In some embodiments, the systems and methods described herein can make determinations with respect to individuals by measuring the subjects' responses to stimuli and comparing the measurements to a reference, such as a baseline measurement. The baseline measurements could be generated from initial testing sessions. Baseline studies can reveal impairments that are acute, chronic, temporary, or permanent in nature. Baseline studies can also demonstrate intact, non-impaired sensory, cognitive, and motor function. The baseline measures also provide data for subsequent identification of the individual. The systems described herein can perform single, repeated, and/or continuous cognitive assessments in order to characterize each individual's current status and any changes in their status with respect to their individual baseline(s), as well as allowing population and norm-based comparisons. The systems facilitate detection of gradual changes and trends, which may be otherwise difficult to identify, and which may reflect degrees of impairment rendering the individual unable to safely and effectively perform designated actions. In addition, detection of significant acute changes may indicate need for urgent medical evaluation and intervention.

In some embodiments, test results can be evaluated with respect to whether they meet and satisfy a set of identity, access, and action-related criteria for granting of permission. The criteria could be pre-established or dynamic in nature. Further, the criteria could be set by the operators of the resources that the permission controls are associated with. The criteria could govern authorized identity, access, and actions. Based on the test results, the systems can take a variety of actions, such as, through connections with hardware and/or software systems, permitting or denying access and/or actions, notifying appropriate people and systems, and/or detaining the individual for further action.

In some embodiments, the testing results provided by the systems and methods described herein can be used by: (i) the subjects to self-identify impairment in order to avoid possible injury and harm during periods of transient intoxication or to prompt health care consultation regarding acute or chronic medical conditions; (ii) employers and/or supervisors to prevent injury, harm, and/or damage; (iii) officials to protect public safety and security (e.g., by preventing individuals with an infectious disease, such as COVID-19, from accessing public locations); and (iv) access control systems to allow or deny access to a location, vehicle, information asset, and so on.

In some embodiments, the systems can be programmed to continuously monitor the subjects as they interact with the resource itself (e.g., a smart device) or through inputs from instruments, such as biometric data acquisition devices. The systems can also be programmed to perform interval monitoring in periodic or episodic fashion. Requirements for retesting can be programmed at predetermined or random times. Retesting subjects after they have been provided at least initially with permission for access and/or action can be beneficial for two reasons: (i) to monitor for the development of an impairment that occurs over time (e.g., fatigue, sleepiness, alcohol, or drugs) and (ii) to prevent an individual other than the authorized individual from gaining access (e.g., preventing an individual that was not the tested subject from driving a car).

As described in further detail below, these systems and methods are based on assessing individuals' cognitive function. Cognitive function includes learning and memory, language, visuospatial, executive, and psychomotor function. Further, cognitive function is frequently underdiagnosed until daily functioning is disrupted. Early detection of cognitive decline can guide intervention to promote retention and, in some cases, improvement in cognitive functioning.

The neurological conditions of delirium (i.e., acute and fluctuating course of inattention, disorganized thinking, and altered level of consciousness) and dementia (chronic or persistent disorder of thinking, remembering, and reasoning) can be differentiated by a number of characteristics, including onset timing, pace of deterioration, duration, course and timing of resolution or permanency, associated attention deficit, level of consciousness, orientation to time/place/person, disturbances of language and speech, preservation or loss of memory, and so on. Although the two conditions can coexist, identifying delirium is important in that it can lead to identification of underlying causes and trigger both preventive and treatment measures.

Analysis of speech and vocal characteristics employed for cognitive assessment include vocal cognitive tasks such as sentence repetition, denomination, picture description, verbal fluency phonemic, verbal fluency sematic, counting backward, and positive/negative/episodic storytelling. Various autonomic nervous system responses could also be used for cognitive assessments. For example, pupil dilation is an autonomic nervous system response to many different kinds of stimuli, including light, smell, taste, a variety of drugs, interest in the subject of attention or arousal, sexual stimulation, uncertainty, decision conflict, errors, or increasing cognitive load or demand. Further, individuals' response time and degree of response tend to change with age. In particular, a lack of pupil dilation in response to olfactory or gustatory stimulation is a good indicator of smell or taste disturbance associated with COVID-19 infection, as described in U.S. patent application Ser. No. 17/167,728.

A variety of different testing modalities can be used to assess cognitive impairment exhibited by subjects. For example, pupillary response to autonomic stimuli, including sensory stimuli (light, sound, smell, taste, pain, etc.), mental effort, and emotion, is a useful means of screening for a number of conditions and states, including effects of substance intoxications, drugs, medications, toxins, brain injury, and/or diseases. Assessment of pupillary response can be used to screen for these and other conditions.

As another example, eye movement characteristics can be a useful tool to help differentiate normal function from that of neurological diseases including mild cognitive impairment (such as that found in dementia, Alzheimer's disease, traumatic brain injury, other neurodegenerative diseases, marked sleepiness, significant fatigue, and substance intoxication). Additional uses include assessments of individuals with autism, depression, schizophrenia, vertigo, congenital nystagmus, and epilepsy. Video-oculo-graphic recordings of eye movements are an objective means for quantitatively assessing ocular motor performance. These findings can be sensitive biomarkers, both as a current “snapshot” of status or as a longer-term history of performance. Testing subjects' eye movement characteristics involves assessing subjects' overall eye tracking, attention to target, dwell time on target, and other measured gaze variables (e.g., fixations, pursuits, saccades, gaze shifts, visual searching, and social cognition). These can be performed with or without specialized head-mounted hardware and can be accomplished without the need to have fixation of the head in place. Eye movement can be recorded as individual images or in video format to which artificial intelligence/machine learning methods can be applied for big data analysis, data mining, and more refined classification of normal and abnormal responses.

As another example, eye tracking characteristics can be used to assess subjects' cognitive impairment. Eye tracking techniques objectively measure, in space and time, both the position of the eyes and their movement. In addition to yielding information regarding oculomotor muscle function, eye tracking allows characterization of target scanning errors including distraction/disinhibition errors, staring/perseverative errors, and order/sequencing errors which allow more refined evaluation of cognitive dysfunction in neurological disorders. The measured quantitative parameters can serve as noninvasive markers for change in cognition and detection of cognitive impairment resulting from drowsiness, fatigue, substance intoxication, injury, and/or disease.

As yet another example, trail-making tests (TMTs) are known as a methodology for neuropsychological assessment of cognitive processes including visual attention, visual search and motor scanning, sequencing and task-switching, psychomotor processing speed, ability to execute a plan of action, as well as higher level cognitive skills such as mental flexibility. TMTs are timed measurements connecting numbers and/or letters in numerical and/or alphabetical order, in forward, backward, or alternating fashion. TMTs reflect cognitive abilities of speed and fluid intelligence and can be employed as a current “snapshot” to identify cognitive impairment and, when compared with baseline determinations, can detect and track deterioration or improvement over time by severity of impairment. In addition, for certain conditions like amyotrophic lateral sclerosis, motor neurons serving ocular function are largely preserved allowing testing otherwise impossible to do with skeletal muscle function.

As yet another example, eye-hand coordination (EHC) tests can be used to assess subjects' cognitive impairment. EHC is the interconnected relationship between visual and manual motor systems. Visually-guided object interaction requires visual detection and motor coordination of the hand to produce intentional, controlled, timed, and accurate movements. Eye-hand coordination testing can be used as a tool to assess neurological disorders and conditions. For example, decline in visual-manual motor functions has been demonstrated in early stages of neurodegenerative disorders such as Alzheimer's disease and Parkinson's disease. Measurement of spatial and temporal deficits can be of use to detect impairment and monitor progression or improvement of the disabling effects of sleepiness, fatigue, substance intoxication, injury, and/or disease.

Other neurophysiological, neuropsychological, and psychomotor tests that could be used to assess whether subjects are exhibiting cognitive impairment and that can be incorporated into a smart device could include, for example: (i) Stroop Color and Word Test, which evaluates selective attention and inhibition control through assessment of the ability to inhibit cognitive interference when processing of a stimulus feature impedes the simultaneous processing of another stimulus feature; (ii) object tracking by vision and/or by hand movement, which assesses fine motor control of eye gaze including following a moving object, scanning the environment for information, directing hand movements, and accurately shifting eye gaze from one target to another; (iii) direction change, which assesses visual working memory by perceiving a visible change in a target image; (iv) visual search, which assesses attention through active scanning of visual environments for specific targets among other distractor objects or features; (v) memory scanning, which assesses general cognitive processing by analyzing storage and retrieval of random information from short-term memory; (vi) flanker test, which assesses spatial selective attention and executive control by analyzing the ability to suppress responses that are inappropriate in specific contexts; (v) digit symbol substitution test, which assesses associative learning through matching of symbols to numbers according to decryption keys; (vi) decision making reaction time, which assesses general alertness and motor speed and the ability to detect, process, and respond to a stimulus by analyzing the time between perception and response; and/or (vii) balance testing, which assesses postural stability in various positions through analysis of body movement and sway. These and other testing techniques can also be used to create individualized dynamic adaptive test results as a basis for identification of the individual.

In some embodiments, the permission control systems described herein can include smart device-based technology in order to assess and monitor subjects. In particular, tablet and smartphone-based technology has been employed to assess cognitive impairment and neurological disorders. Although most medical investigations for characterization of cognitive and other neurological abnormalities involve invasive, time-consuming, expensive, and sometimes difficult to access technologies, smart device-based assessments can be performed in the comfort of one's own home or at mass testing kiosks. In particular, smart device-based technology makes assessment of those who are older, infirm, and have physical disabilities easier, which is becoming increasingly important as the number of people diagnosed with cognitive impairment is growing rapidly as the population ages and as more individuals sustain concussive head injuries. It has been shown that the majority of older adults own or have ready access to smart devices.

Smart device technology can include testing for immediate recognition, semantic memory, categorization, subtraction, repeating backward, clock drawing, cube copy, cube rotation, pyramid rotation, trail making delayed recognition tests, symbol matching tasks, memory tasks, object matching tasks to assess a variety of aspects of cognitive function, including concentration, memory, and visuospatial function.

Various embodiments of the systems and methods described herein can assess subjects' decline in cognitive function through changes in physical movement. Further, GPS data focusing on geographic area and perimeter of activities of daily life could be used as indicators of both physical and cognitive function. The area and perimeter coverage within and outside the home can be used to distinguish between healthy people and those with cognitive impairments like dementia. Further, accelerometer-derived gait velocity can yield information about disorders of affective status such as depression. Assessment of fine motor skills from tapping on the screen; movement of right, left, or both hands in time with audio signals; and measures of tap response time, rhythm, and contact duration, and inter-hand divergence are used to assess for dementia and mild cognitive impairment.

Accordingly, a smart device-based system and methodology to detect and characterize subtle and non-subtle cognitive and other neurological disturbances in individuals in domains of time and space. The systems can utilize single and multiple (over-time) determinations of neuropsychological and psychomotor assessment with comparison to the individual's baseline determinations as well as to population-derived values to detect and characterize incipient or established abnormalities of response. These results are compared with the criteria for the actions to determine whether or not the individual should be granted permission to perform the actions or access the desired assets. These findings can then trigger notification of the individual to avoid actions which may confer risk of injury and harm and also to seek professional medical attention for evaluation and further workup and treatment as appropriate. The findings can trigger notification of employers, supervisors, and/or security personnel to prevent injury, harm, and/or damage, and also notification of officials to protect public safety and security. The findings can also trigger referrals to healthcare for evaluation of possible diseases and disorders.

Many physical and virtual environments already use electronic interfaces to provide access or permit other functions. For example, electronic keys, badges, credit cards, and specialized devices link to cars, doors, cash registers, toll booths, and computers. Cars use standardized electromagnetic links between electronic keys and vehicles both to unlock doors and to permit the vehicle to be started, and even identify the user, for example, to adjust the seats or change the driving characteristics. Thus, smart devices are already able to directly provide, or block, access to all manner of situations depending on results of the screening tests. In other cases, the smart device can use a variety of secure mechanisms to notify another device or person of the results of the screening so they can take appropriate actions. Accordingly, the various embodiments of permission control systems described herein could be incorporated into such physical and/or virtual environments (or the electronic interfaces therefor).

### Permission Control Systems

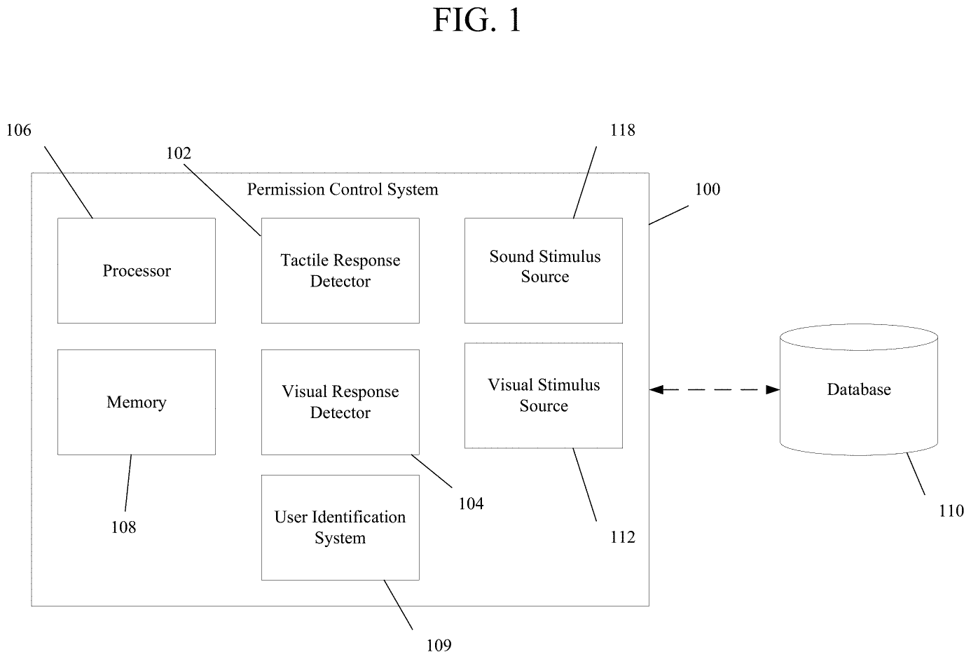

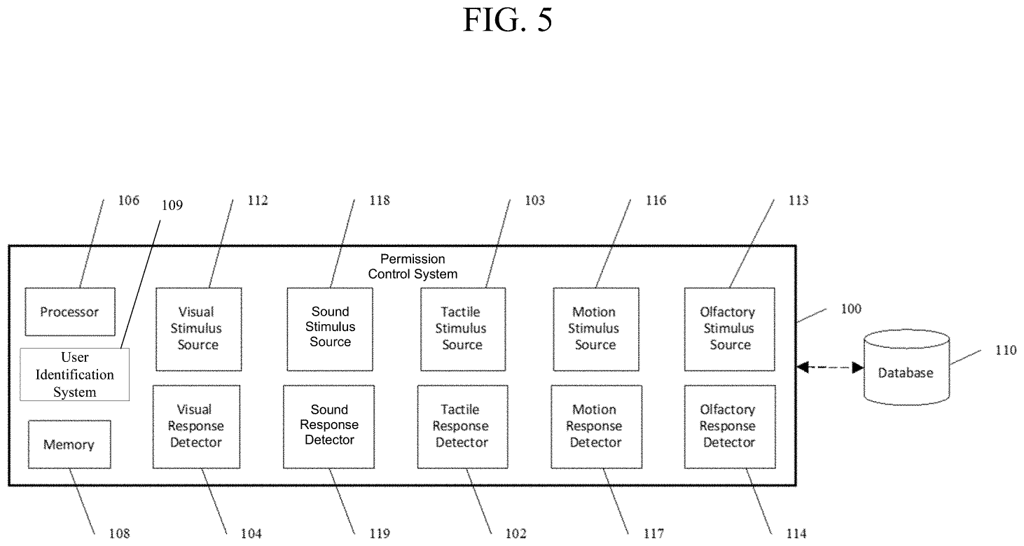

In one embodiment, a permission control system 100, such as are shown in FIG. 1 and FIG. 5, can include a visual stimulus source 112, a tactile response detector 102, and a visual response detector 104. The visual stimulus source 112 can include a standalone display screen or a display integrated into another device (e.g., a display of a smartphone). In one embodiment, the visual stimulus source 112 and the tactile response detector 102 can be integral to each other. For example, the visual stimulus source 112 and the tactile response detector 102 can be embodied as a touchscreen (e.g., a capacitive touchscreen). In one embodiment, the visual response detector 104 can include a camera or an image sensor. The visual response detector 104 can have sufficient resolution and other characteristics necessary to be able to detect the movements of a subject's eyes or portions thereof (e.g., the pupil) when positioned within a threshold distance to the subject.

The permission control system 100 can be programmed or otherwise configured to provide a visual stimulus to a subject and track, monitor, or record the subject's response to the visual stimulus. Based on the subject's tracked response to the visual stimulus, the permission control system 100 can be programmed or otherwise configured to make a determination as to whether the subject has findings compatible with a neuropathological condition, such as Long COVID or dementia. The visual stimulus provided to the subject could include a series of dots or patterns, icons, alphanumeric characters, or any other markers that can be visually identified and tracked by subjects. In various embodiments, the visual stimulus or portions thereof can move across the visual stimulus source 112, disappear and/or appear at various points on the visual stimulus source 112, change color, change shape, change visual perspective (e.g., rotate), and otherwise change in visually detectable manners. The permission control system 100 can track a variety of different types of responses by a subject to the stimulus, including, for example, ocular responses, physical responses, or autonomic responses by the subject. In one embodiment, the permission control system 100 can be configured to track eye movements by the subject in response to movements or changes by the visual stimulus. In another embodiment, the permission control system 100 can be configured to track the ability of the subject to perform a trail-making test. As noted above, a trail-making test is a timed measurement of a subject's ability to connect numbers, letters, or other visual markers in a particular order (e.g., numerical or alphabetical order). A trail-making test can further task the subject with connecting the visual markers in a variety of different manners (e.g., in a forward, a backward, or an alternating fashion). The permission control system 100 can track the subject's response(s) to the visual stimulus via the tactile response detector 102, the visual response detector 104, or a combination thereof depending upon the particular response being tracked thereby. For example, in embodiments where the response being tracked is the subject's eye movements, the permission control system 100 can utilize the visual response detector 104 to track the characteristics of the subject's eyes. As another example, in embodiments where the response being tracked is the subject's ability to perform a trail-making test, the permission control system 100 can utilize the tactile response detector 102 to track the subject's response to the trail-making test.

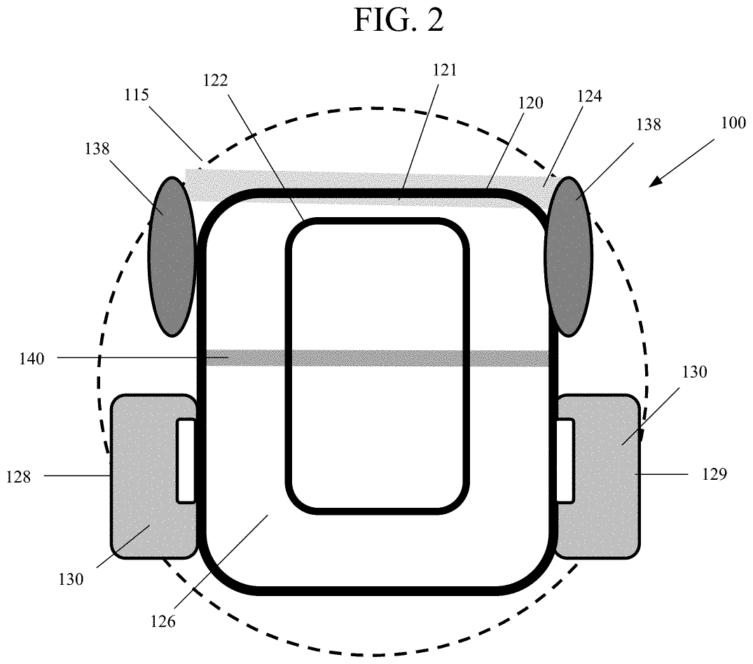

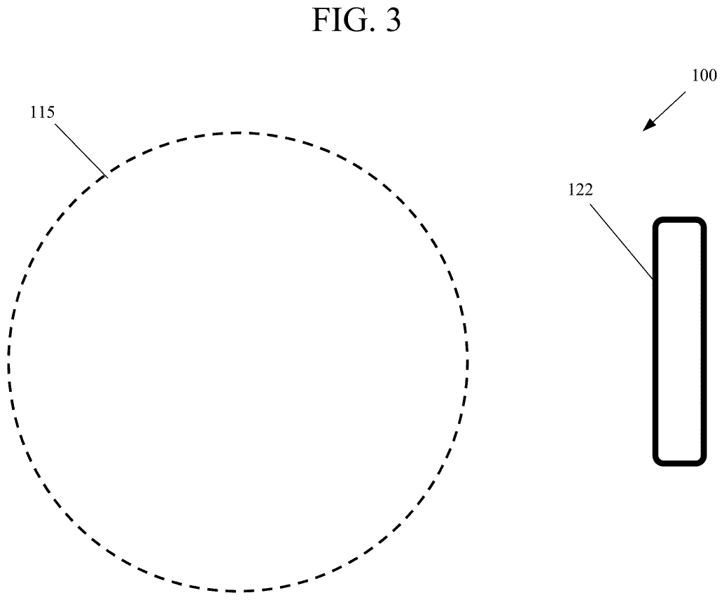

The visual response detector 104 could include standalone sensing devices or be incorporated into another device (e.g., a mobile device 122, as in the embodiments shown in FIGS. 2 and 3) or system. Further, in some embodiments, the visual response detector 104 could include one sensor or a set of sensors (i.e., a sensor assembly). The permission control system 100 can be configured to execute various processes, such as those described below, to screen individuals based on their response or responses to stimuli provided by the permission control system 100. In one embodiment, the permission control system 100 can further include a processor 106 coupled to a memory 108 for storing data, including logic or instructions embodying processes to be executed by the processor.

The visual response detector 104 can be configured to capture images or video of a subject in sufficient detail such that the subject's eye movement response to the visual stimulus can be measured and, thus, quantified. In other words, the visual response detector 104 can be configured to capture images or video in a sufficiently high resolution and with sufficient clarity such that image processing algorithms can identify the subject's eyes (or portions thereof, such as pupils) and measure changes associated therewith. In various embodiments, the ocular response measured by the visual response detector 104 could include a change in the size (e.g., diameter or area) of the subject's pupil or pupils, timing information (e.g., hesitancy or delay in the pupil's movement), and other pupillary parameters. For example, the visual response detector 104 could be used to take a first measurement of a characteristic of the subject's eyes and take a second measurement of the characteristic after the subject has been provided the visual stimulus or after the initially provided visual stimulus has been changed by the permission control system 100 (e.g., has moved, disappeared and reappeared at a different location on the visual stimulus source 112, changed in shape, or changed color). Accordingly, the subject's response to the visual stimulus could include the difference between the different measurements of the ocular characteristic.

In one embodiment, the permission control system 100 could further include a sound stimulus source 118. The sound stimulus source 118 can be used to deliver instructions, produce audio signals for tapping cadence, be an element of the stimulus, or provide distractions as necessary for the testing.

The permission control system 100 can be embodied as a variety of different objects, devices, or systems. In one embodiment, the permission control system 100 could include a mobile device (e.g., a smartphone) and the processes executed thereby could include an app. In this embodiment, the permission control system 100 could be beneficial by allowing individuals to self-screen for a particular condition or set of conditions using their own mobile device. In some embodiments, the visual response detector 104 could be embodied as an accessory or dongle that is connectable (either wirelessly or via a wired connection) or attachable to the mobile device. In other embodiments, the visual response detector 104 could include the onboard camera of the mobile device. Other embodiments could be suitable for screening individuals for entry to potentially crowded locations (e.g., schools, airports, or stadia). In one such embodiment, the permission control system 100 could include a kiosk or station that includes the visual stimulus source 112 for providing the visual stimulus to subjects within the kiosk and the tactile response detector 102 and/or visual response detector 104. In this embodiment, the permission control system 100 could be beneficial by allowing individuals to be screened for potential abnormalities (e.g., such as those associated with Long COVID or COVID-19 generally) prior to being permitted entry into a location or allowed to perform an action or activity. An abnormal response could be used as one of the tools to decide whether individuals should be permitted access to a venue location or allowed to perform an action or activity, or require additional screening, thereby potentially avoiding significant adverse consequences (e.g., disease transmission events).

The permission control system 100 can further include or be communicably connected to a database 110. The database 110 could include a local database 204 and/or a remote database 206, as described below. In one embodiment, the database 110 could be stored locally (i.e., in the memory 108). In another embodiment, the database 110 could be remote from the permission control system 100. In this embodiment, the database 110 could be stored in a cloud computing storage system (e.g., Amazon Web Services), a remote server, and other such remote systems. The database 110 can be configured to store information including user parameters and settings, such as the user's previously calibrated responses. The user parameters could be embodied as a user profile, for example. The user parameters could include previously recorded values or measurements associated with the response measured by the permission control system 100. The recorded parameters can be used to define a characterized or default response by the subject to the stimulus, which can in turn be used by the permission control system 100 to determine if and when the subject's measured response deviates from this characterized or default response by the subject. Accordingly, the permission control system 100 can determine when there has been a change in the subject's response to the stimulus, which could indicate that the subject has a condition that is screened by the permission control system 100. The characterized or default response could be used to define various thresholds or ranges that could be used to determine whether the subject has passed or failed the screening. Accordingly, the permission control system 100 can be configured to take measurements (e.g., via the visual response detector 104 and/or tactile response detector 102) associated with the subject's response to the stimulus, retrieve a user profile associated with the subject (e.g., from the database 110), and determine whether the subject has passed or failed the screening based on a comparison between the measurements of the response and the user profile parameters or a reference. For example, the permission control system 100 can be configured to provide an eye tracking test and/or a trail-making test to the subject and measure the subject's response (i.e., the ability to properly track a moving visual stimulus with the eyes or respond to a static visual stimulus with tactile response) thereto. If there is a significant deviation from the subject's ability to perform the trail-making test relative to a reference or baseline (e.g., the stored, pre-characterized performance on the trail-making test associated with the subject or a universal characterized response), then the subject may be suffering from a neuropathological condition. Accordingly, the permission control system 100 could prompt the subject regarding the need for medical evaluation (such as a physician evaluation and/or testing including COVID-19 test) and/or suggest that the individual take corresponding appropriate precautions (e.g., self-quarantine or isolation). Conversely, if there is no significant deviation from the subject's ability to perform the trail-making test as compared to the reference or baseline, then the subject may not be suffering from such a neuropathological condition. Accordingly, the permission control system 100 could grant the subject the permission(s) sought thereby. In some embodiments, the permission control system 100 can further record the subject's response(s) to the provided visual stimulus. The recorded responses could be used to further characterize the subject's baseline response characteristics, aggregated with records of other subjects (e.g., in the database 110) to further characterize universal or population-wide response characteristics, and so on.

The permission control system 100 can further be configured to account for various secondary factors and be calibrated for each individual subject. For example, the permission control system 100 may need to be calibrated to determine the baseline or expected response characteristics (e.g., eye movement hesitancy or degree of dilation in response to various visual stimuli or the sequence of objects the subject's eyes focus on) exhibited by the subject. Once the baseline or expected response characteristics are determined, measurements in the subject of those characteristics by the permission control system 100 can be used to distinguish between usual/normal responses, potential neuropathological conditions and other abnormalities.

In one embodiment, the permission control system 100 can be configured to determine the amount of light in the subject's environment (e.g., via the visual response detector 104) and, accordingly, account for the amount of light employed to determine the subject's response to the screening. The amount of ambient or environmental light can be an important factor because it can affect the ability of the subject's eyes to properly identify and distinguish between various visual stimuli, the contraction and dilation of the subject's pupils, and so on. In particular, a brightly lit ambient environment could decrease the pupil opening and, because the pupil is constricted, it may not dilate normally in response to particular visual stimuli. Conversely, in a dimly lit ambient environment, it may be more difficult for the subject's eyes to track the movement of the visual stimuli, or the permission control system 100 may be unable to properly detect the subject's ocular response characteristics to the visual stimuli. Thus, in some embodiments, the permission control system 100 can measure the amount of environmental light and recommend or affect adjustments for appropriate screening, such as blocking environmental light. In some embodiments, the permission control system 100 can additionally be configured to control the amount of light in the test environment, such as by activating or controlling lights in the test environment.

In one embodiment, the permission control system 100 could be configured to determine whether the subject has certain conditions, whether preexisting or temporary, that could affect its measurements. For example, the permission control system 100 could automatically detect the presence of various indicators (e.g., cataracts), retrieve subject information (e.g., electronic medical records) from a database, or prompt the user to enter such information. This information is important because some indicators (such as cataracts) could affect pupillary response. Pupillary responses may be affected in different ways by different indicators. Thus, in some embodiments, the permission control system 100 could be configured to detect and differentiate among such indicators. The permission control system 100 could be configured to incorporate the presence of these indicators into the determination of the likelihood that the decrease or lack of ocular response is related to the tracked conditions. In addition, the permission control system 100 could be configured to recommend additional screenings in the event that more determinations or more time would be advantageous in achieving a successful screening.

In some embodiments, the permission control system 100 could be configured to account for a variety of other events or subject-specific or environmental conditions. For example, the permission control system 100 could be configured to detect when the user was startled at the time of the test (e.g., due to a loud sound or bright flash) and adjust environmental conditions or recommend that the subject be retested. In one implementation, the permission control system 100 could ask the subject one or more questions, such as, “Are you currently suffering from a headache or a lack of sleep?”, prior to beginning the screening test and act accordingly based on the subject's responses (e.g., recommending that the test be postponed). As another example, the permission control system 100 could be configured to detect (e.g., via the visual response detector 104) whether there was any motion within the subject's field of vision that could have distracted the subject.

All of the various data associated with the subject that are discussed above, such as the subject's amount of ocular response characteristics at various lighting levels, the subject's response to various tests (e.g., accuracy or timing in performing trail-making tests), and so on, could be stored in a user profile associated with the subject. As discussed above, this data can be stored by and/or retrieved by the permission control system 100 (e.g., from the database 110) at the time of the screening test to assist in the determination of positive or negative screening results.

One embodiment for the permission control system 100 is shown in FIG. 2. In this embodiment, the permission control system 100 could include a headpiece 120 that can be worn on the head 115 of the subject. In the illustrated embodiment, the headpiece 120 includes a holder 121 that is configured to hold a mobile device 122 (e.g., a smartphone or another smart device) that includes a visual response detector 104 (e.g., a camera). The holder 121 can be configured to hold the mobile device 122 such that the visual response detector 104 is oriented towards the subject's face when the headpiece 120 is worn by the subject. The mobile device 122 executing the app can be used to guide the subject through the screening steps with audio, images, text, or combinations thereof. Accordingly, in one implementation, users could place their mobile device 122 in the holder 121, and the mobile device can in turn execute an app stored thereon that performs the screening test, as described herein. In another embodiment, the detector 104 could be integral to the headpiece 120. The headpiece 120 can include one or more straps 124 or other securement devices for securing the headpiece to the subject's head 115 and keep the mobile device 122 in a fixed relationship to the subject's face.

In one embodiment, the headpiece 120 could define a partially or fully enclosed chamber 126 that is configured to provide a fixed environment suitable for the screening test for the subject. The headpiece 120 could further include an air inlet 128 (which can further include a filter 130, such as a P100 filter) and a corresponding outlet 129 for allowing the subject to exhale. The screening test could be activated manually by the subject or automatically by the permission control system 100 (e.g., by the software app running on a mobile device 122). In still other embodiments, the headpiece 120 could include earphones 138 to control or reduce environmental noise and direct sounds from the mobile device 122 or sensor to the subject. Such embodiments can be beneficial because they provide a controlled environment for the performance of the screening test, which can increase reliability of the results.

Another embodiment of the permission control system 100 is shown in FIG. 3. In this embodiment, rather than using the headpiece assembly described above with respect to FIG. 2, the subject could instead hold their mobile device 122 in close proximity to his or her face (or rest the mobile device 122 in an appropriate location). In this embodiment, the visual response detector 104 could include the onboard camera of the mobile device 122, and the tactile response detector 102 could include the capacitive touchscreen thereof. In this embodiment, the relationship between the subject's head 115 and the mobile device 122 is not fixed, so the mobile device (or the software app executed thereby) can therefore be configured to compensate for motion of the subject relative to the mobile device. Such an embodiment can be beneficial because of its ease of use. In particular, such an embodiment does not require a substantial number of components or for the subject to wear a head assembly or otherwise be within a fixed environment.

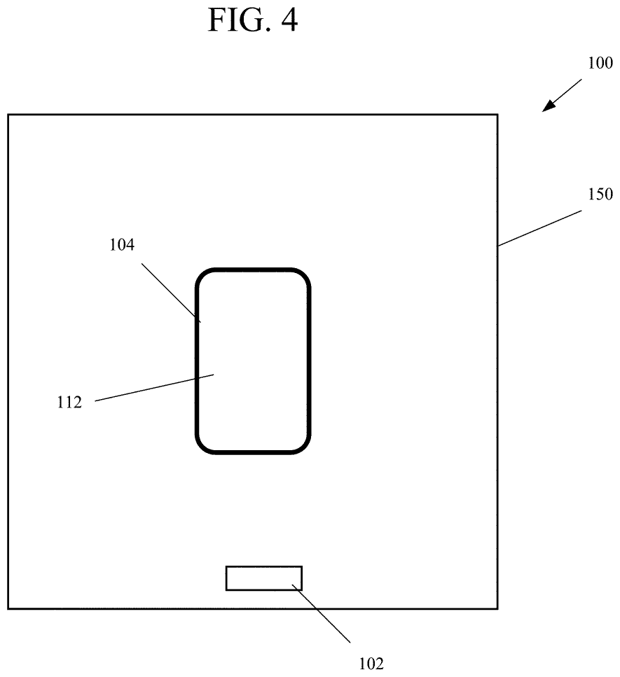

Yet another embodiment of the permission control system 100 is shown in FIG. 4. In this embodiment, the permission control system 100 is embodied as a high-throughput system that could be suitable for screening at airports, stadia, and so on. In particular, this embodiment of the permission control system 100 can include an enclosure 150 into which the subject can enter. The enclosure 150 could be an enclosure that is environmentally controlled, for example. The enclosure 150 could include a complete or partial enclosure (e.g., from the waist up). In such an embodiment, the subject enters the enclosure 150 and faces the visual stimulus source 112, which can further include the tactile response detector 102 (e.g., a capacitive touchscreen). As noted above, the visual response detector 104 could include a camera or an image sensor, for example. The visual stimulus source 112, visual response detector 104, and/or tactile response detector 102 could be positioned on a wall of the enclosure at a height suitable for visualizing individuals' faces and/or being readily reached by the individuals, for example, or at adjustable heights to optimize the relationship to the user. The visual stimulus source 112 could provide appropriate instructions to the subject (e.g., where to stand), what information to provide to the permission control system 100 (e.g., whether the subject has any relevant information that may inform results of screening), or when to exit. The visual stimulus source 112, visual response detector 104, and/or tactile response detector 102 could include a smart device or a specialized sensor apparatus. The visual stimulus source 112, visual response detector 104, and/or tactile response detector 102 could be integral to or otherwise located within the enclosure 150.

Another embodiment of the permission control system 100 is shown in FIG. 5. In this embodiment, the permission control system 100 could be embodied as a mobile device (e.g., a smartphone). This embodiment uses emerging capabilities of mobile devices to communicate more effectively with people, especially those with varying disabilities, such as hearing, motion and visual challenges, and for additional screening capabilities. In this embodiment, the permission control system 100 is configured to provide a variety of different stimuli to the subject and detect the responses by the subject thereto. In particular, the permission control system 100 can include a visual stimulus source 112, a sound stimulus source 118, a visual response detector 104, and a tactile response detector 102, as described above. In this embodiment, the permission control system 100 can further include one or more of a sound response detector 119, a tactile stimulus source 103, an olfactory stimulus source 113, a motion stimulus source 116, a motion response detector 117, and an olfactory response detector 114, including any combination thereof. The sound response detector 119, such as a microphone, is configured to detect and measure speech and other sounds from the subject, such as verbal responses, slurring of speech, heart beats, and bodily sounds. In some embodiment, the sound response detector 119 can further be configured to detect and measure potentially interfering sounds from the environment. The tactile response detector 102 is configured to detect and measure details of touch, such as, how hard a subject is pressing, how many fingers are touching the detector, details of motion, and uneven touch pressure such as from a tremor. The tactile stimulus source 103 is configured to create the impression of response to touch, such as depressing a virtual key by a subtle motion. The motion stimulus source 116 is configured to generate small motions, such as vibrations, which can be used to assess the ability to detect motion stimuli as well as give feedback to a subject, such as used to get attention when a smart phone is in silent mode. The permission control system 100 can further be programmed or otherwise configured to perform speech-to-text translation (e.g., via software) executed by the processor 106) to detect and measure the appropriateness and accuracy of verbal responses derived from the speech of the subject. The motion response detector 117, such as an accelerometer, is configured to detect and measure changes in motion exhibited by the subject, such as tremors, startle responses, uneven motions (e.g., due to Parkinson's and other causes), and heartbeats. The method can include other stimuli and detectors, such as electrical stimuli and the associated detectors. The olfactory stimulus source 113 is configured to generate an olfactory stimulus and an olfactory stimulus detector 114 that is configured to detect and measure the same, such as is disclosed in U.S. patent application Ser. No. 17/167,728, now U.S. Pat. No. 11,083,405, titled SYSTEMS AND METHODS FOR SCREENING SUBJECTS BASED ON PUPILLARY RESPONSE TO OLFACTORY STIMULATION, filed Feb. 4, 2021, which is hereby incorporated by reference herein in its entirety.

In some embodiments, the permission control system 100 can include a user identification system 109 that is configured to determine whether the individual interacting with the permission control system 100 (e.g., attempting to take a screening test) is the correct user or one of the correct users associated with the permission control system 100. The user identification system 109 could be configured to make use of dynamic adaptive biometrics, such as are described below.

As noted above, in one embodiment, the permission control system 100 can be embodied as a mobile device 122. In other embodiments, the permission control system 100 could be embodied as a combination of devices and/or systems that are communicatively coupled. For example, the permission control system 100 could include a mobile device 122 that is coupled to a remote computing system (e.g., a server or a cloud computing system). In such embodiments, various steps, aspects, or techniques described above can be collectively executed by or between the devices and/or systems.

In various embodiments, the permission control system 100 could be associated with a variety of different devices, locations, objects, content, actions, activities, and so on in order to control access and/or action thereto. In various embodiments, the permission control system 100 could be used to control access and/or action regarding physical and/or virtual resources. In one embodiment, the permission control system 100 could be associated with an automobile and the permission control system 100 could control users' ability to turn on and/or drive the automobile. This embodiment could be beneficial because it could be used to prevent individuals from driving the automobile only if they are not exhibiting signs or symptoms of cognitive impairment (e.g., due to alcohol consumption). In another embodiment, the permission control system 100 could be associated with a physical location (e.g., a security checkpoint in a building). In this embodiment, the permission control system 100 could provide an alert to security personnel indicating whether the individual could be suffering from cognitive impairment. This embodiment could be beneficial because cognitive impairment is a symptom of some infectious diseases (e.g., brain fog in COVID-19). Accordingly, the permission control system 100 could be used for screening for the potential presence of a particular infectious disease in individuals that are seeking to access a premises for the potential presence of a particular infectious disease. In other embodiments, the permission control system 100 could be associated with smartphones, computers, door access panels, and so on.

In one embodiment, the permission control system 100 could be incorporated into a control system (e.g., an access control system) for a location, device, or resource. For example, the permission control system 100 could be incorporated into a control system for a vehicle. In one such embodiment, the permission control system 100 could be interfaced with the vehicle's ignition control module to control the user's ability to start the vehicle. In another embodiment where the vehicle is an autonomous vehicle, the permission control system 100 could be interfaced with the vehicle's autonomous driving control system to control whether and how the vehicle is driven. In this embodiment, the permission control system 100 could, via the autonomous driving control system, cause the vehicle to switch into an autonomous driving mode or pull over to the side of the road and/or slow down (e.g., when the permission control system 100 determines that access should be revoked from the driver, such as if the system 100 is detecting that the driver is potentially suffering from cognitive impairment due to drugs or alcohol). As another example, the permission control system 100 could be incorporated into a building security system. In one such embodiment, the permission control system 100 could be interfaced with the building security system's electronic lock system. Accordingly, the permission control system 100 could, via the electronic lock system, deny or revoke access to the building or various locations therein. In an alternative embodiment, the permission control system 100 could be communicably coupled to a control system for a location, device, or resource. For example, the permission control system 100 could be communicably coupled to an ignition control module of a vehicle, an autonomous driving control system of a vehicle, or an electronic lock system of a building. Although not incorporated directly into the control systems, the permission control system 100 could otherwise function as described above for the other embodiments.

Cognitive Assessment & Permission Control Techniques

As described above, the various embodiments of the permission control system 100 can be configured to provide an individual with a variety of different neurophysiological stimuli in order to assess the individual's response(s) to the stimuli and thereby determine whether the individual is suffering from cognitive impairment. The permission control system 100 can further control whether the individual is provided the necessary permission(s) (e.g., to access the location or perform an action) based on whether the system 100 has determined that the individual could be suffering from cognitive impairment. In various embodiments, the permission control system 100 can provide a variety of different stimuli to users, including the testing techniques and stimuli disclosed in U.S. patent application Ser. No. 17/528,417, titled SYSTEMS AND METHODS FOR SCREENING SUBJECTS FOR NEUROPATHOLOGY ASSOCIATED WITH A CONDITION UTILIZING A MOBILE DEVICE, filed Nov. 17, 2021, which is hereby incorporated by reference herein in its entirety. In various embodiments, the permission control system 100 could provide a single neurophysiological stimulus or a combination of neurophysiological stimuli. In various embodiments, the neurophysiological stimulus provided by the permission control system 100 could include a trail-making test, a finger tapping test, or any other type of stimulus or testing technique described in U.S. patent application Ser. No. 17/528,417. In embodiments where the permission control system 100 is providing neurophysiological stimuli embodied as a test (e.g., a trail-making test or a finger tapping test), the neurophysiological stimuli could be provided via the visual display source 112 (e.g., a display screen). In embodiments where the permission control system 100 is at least partially embodied as a smartphone app, the neurophysiological stimuli could be provided as graphical elements displayed via the app on a subject's smartphone.

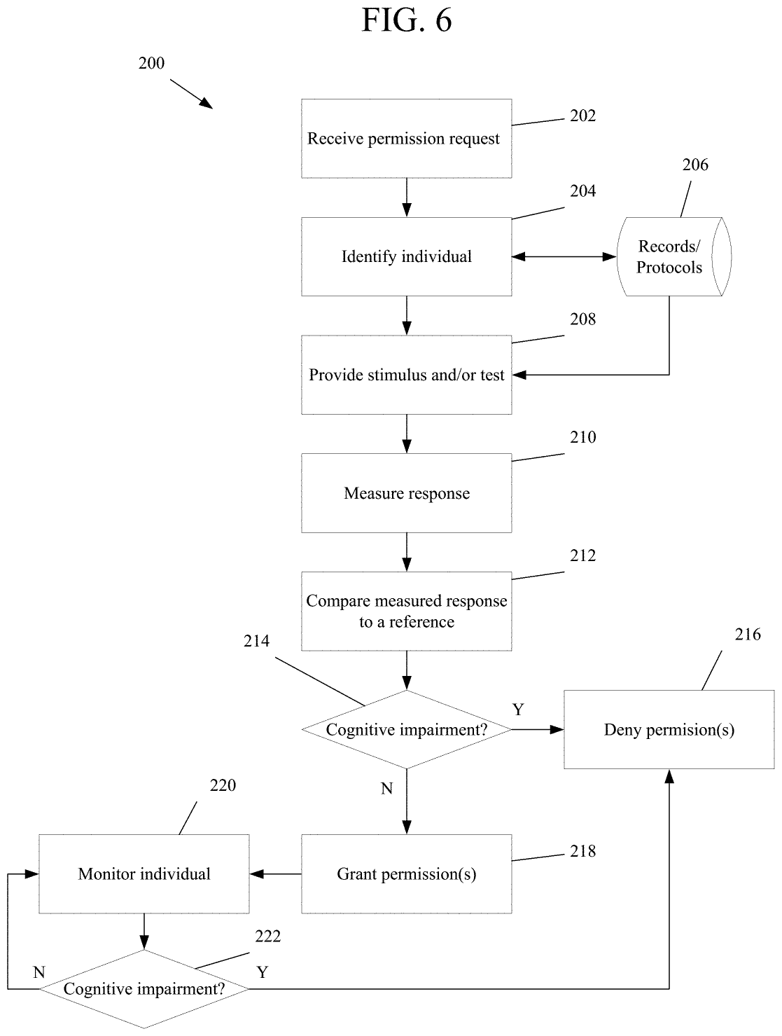

In one embodiment, the permission control system 100 described above can be configured to execute various processes for assessing whether individuals are suffering from any cognitive impairments and manage the individuals' permission controls accordingly. One example of such a process 200 is shown in FIG. 6. In the following discussion of the process 200, reference should also be made to FIG. 1. In one embodiment, the process 200 can be embodied as instructions stored in a memory 108 that, when executed by a processor 106, cause the permission control system 100 to perform the process. In various embodiments, the process 200 can be embodied as software, hardware, firmware, and various combinations thereof. In various embodiments, the process 200 can be executed by and/or between a variety of different devices or systems. For example, one or more steps of the process 200 could be executed by a mobile device of the permission control system 100 and one or more of the remaining steps of the process 200 could be executed by a cloud computing system or another such remote computer system. In various embodiments, the permission control system 100 executing the process 200 can utilize distributed processing, parallel processing, cloud processing, and/or edge computing techniques. The process 200 is described below as being executed by the permission control system 100; accordingly, it should be understood that the functions can be individually or collectively executed by one or multiple devices or systems.

As generally described above, the permission control system 100 can provide various tests to an individual to determine whether the individual could be suffering from a cognitive impairment. In some embodiments, the permission control system 100 can provide a default or predetermined test or set of tests to an individual. In some embodiments, the permission control system 100 can select one or more tests to be provided to the subject. For example, the permission control system 100 executing the process 200 can determine which test or tests to run, i.e., which test or tests are to be provided to the individual. The permission control system 100 can determine which test or tests to provide to the individual based on a variety of different factors, including previous tests provided to the subject or which neuropathological conditions the subject is being screened for. The tests can include, for example, an eye tracking test, a trail-making test, or an eye-hand coordination test.

Accordingly, the permission control system 100 executing the process 200 can receive 202 a permission request (i.e., a request to access a particular device, location, venue, or content) from an individual or user to access a resource and/or perform an action. In various embodiments, the resource could include a physical object (e.g., an automobile, a smartphone, or a building) or a virtual object (e.g., content in a smartphone app). The permission request could take a variety of different forms. In various embodiments, the permission request could include permission to perform an action or activity. In one embodiment, a permission request could include an individual logging into (or attempting to log into) a computer. In another embodiment, a permission request could include a request for access to a conference call. In yet another embodiment, a permission request could include a request to send an electronic message to another individual or computer. In yet another embodiment, a permission request could include a request from a scheduling program to a driver to take a vehicle to a location.

In response to receiving 202 a permission request, the permission control system 100 can identify 204 the individual associated with the permission request. In one embodiment, the permission control system 100 could make use of dynamic adaptive biometric techniques to identify 204 the individual, such as are disclosed in U.S. patent application Ser. No. 17/565,668, titled SYSTEM AND METHODS FOR HUMAN IDENTIFICATION OF INDIVIDUALS WITH COVID-19 UTILIZING DYNAMIC ADAPTIVE BIOMETRICS, filed Dec. 30, 2021, which is hereby incorporated by reference herein in its entirety. In another embodiment, the permission control system 100 can identify 204 the individual by prompting the individual to enter a password associated with the individual's account or profile associated with the permission control system 100. In other embodiments, the permission control system 100 can identify 204 the individual based on a badge, a driver's license, a photograph, an electronic key, or other forms of identification. In some embodiments, the identification 204 of the individual could incorporate a variety of different information, including, for example, an electronic toll booth ID, computer ID, URL, GPS location, QR code, or photograph of a building or sign.

In some embodiments, the permission control system 100 can retrieve 206 data (e.g., records, protocols, and/or other testing criteria) associated with the individual. The data could be retrieved from a database 110, such as is shown in FIG. 1, for example. The data could include the criteria that need to be met for the permissions to be granted by the permission control system 100. The retrieved data could include a particular stimulus or combination of stimuli that are associated with testing the particular individual, reference or baseline measurements associated with the individual for the different stimuli, and so on. For example, there may be a particular neurophysiological stimulus that is particularly suited for testing of the individual based on their particular characteristics and/or the permission control system 100. In some embodiments, the data could be retrieved 206 from a variety of different databases or other sources. For example, the data could be retrieved 206 from an employer database of employees. Further, the criteria executed by the permission control system 100 could vary for each employer and/or employee (e.g., vaccination status may be more crucial for some types of employees, such as doctors and nurses, and less crucial for other types of employees, such as warehouse laborers). The retrieved data could further include vaccination records or medical records (e.g., whether the person has had COVID-19). All of this retrieved data could be incorporated into what types of stimuli/tests are provided to the subject and the criteria used by the permission control system 100 to control the permissions granted thereby.

Accordingly, the permission control system 100 can provide 208 a stimulus (e.g., a neurophysiological stimulus) to the individual. In one embodiment, the provided stimulus could include a trail-making test, a finger tapping test, and so on. Further, the permission control system 100 can measure 210 the individual's response to the stimulus (e.g., the individual's performance on the trail-making test or the finger tapping test), compare 212 the measured response to a reference, and accordingly determine 214 whether the individual is exhibiting signs or symptoms of impairment based on the comparison between their performance on the test relative to the reference. In one embodiment, the reference could include a default value, such as a preprogrammed value associated with the given stimulus. In another embodiment, the reference could include a characterized response by the individual to the stimulus (e.g., an average performance score for the test). For example, the screening system 100 could be programmed to store a number of response measurements by the individual and characterize the normal range of responses of the individual for the given stimulus. In yet another embodiment, the reference could include characterized responses of a population of individuals to the stimulus (e.g., an average performance score by a particular population of individuals for the test). In this embodiment, data from a population of users could be pooled and analyzed to characterize the normal or baseline range of responses for particular stimuli. In one embodiment, the permission control system 100 can determine whether the individual is exhibiting cognitive impairment based on whether the measured neurophysiological response differs from the reference response by a threshold.

If the permission control system 100 determines 214 that the individual is exhibiting cognitive impairment, the system 100 can accordingly deny 216 the permission(s) sought by the individual. In various embodiments, the denial 216 of the permissions could include the individual being locked out from accessing the sought resource or an alert that is provided to personnel that the individual is to be denied access to the resource. For example, in an embodiment where the permission control system 100 is associated with an automobile, the permission control system 100 could prevent the automobile from starting or otherwise prevent the automobile from being driven by the individual. As another example, in an embodiment where the permission control system 100 is associated with a building, the permission control system 100 could prevent the individual from accessing the building (e.g., by not unlocking a door). If the permission control system 100 determines 214 that the individual is not exhibiting cognitive impairment, the system 100 can accordingly grant 218 the permission(s) sought by the individual.

In one embodiment, the permission control system 100 could deny 216 or grant 218 permission(s) further based on a set of criteria. The criteria could include, for example, legal mandates, employer requirements, venue requirements (e.g., concerts, stores, or offices), and so on. The criteria could also include, for example, licenses (e.g., driver's licenses or medical licenses), certificates, training certifications, vaccination statuses, drug test results, a level of knowledge or skill required for a position, the amount of access necessary to a facility or resource, virtual access (e.g., passwords or digital keys), visitor status (e.g., whether the individual is simply visiting a facility or is otherwise temporarily accessing a location or resource), limitations (e.g., whether the individual requires glasses, whether the individual is allowed to drive at night, or whether the individual is on medication), or whether the individual has limited abilities due to a physical or mental condition (e.g., due to pregnancy or an injury). For example, a set of criteria for accessing a certain environment could dictate that only individuals that are exhibiting substantial cognitive impairment be denied permission to access the environment, whereas another set of criteria for accessing another environment could dictate that individuals exhibiting any degree of cognitive impairment be denied permission(s). In some embodiments, the criteria can be dynamic or otherwise change over time.

In one embodiment, the permission control system 100 can further monitor 220 the individual as they continue to interact with the permission control system 100 and/or the accessed resources. If the permission control system 100 determines 222 that the individual exhibits new signs of cognitive impairment, the permission control system 100 can revoke 216 access to the resource. In some embodiments, the permission control system 100 could monitor the subject without providing any further stimuli based on how the subject continues to interact with the system 100. In particular, some neurophysiological responses could be observed without any additional stimuli, including eye motion or pupil dilation, as the subject continues using the resource. For example, in embodiments where the permission control system 100 is associated with a car, as the individual is driving a car, the permission control system 100 could detect whether the individual is getting intoxicated or is suffering from drowsiness based on slow eye motion, whether the subject is closing his or her eyes, whether the subject is looking away from the road for too long, and so on. In some embodiments, the permission control system 100 could provide new or additional stimuli to the subject as the subject is making use of the permission(s) granted by the system 100. For example, in embodiments where the permission control system 100 is associated with a car, the system 100 could provide an additional stimulus when the subject is at a stop light or when the car is in an autonomous driving mode and assess the subject's response to the additional stimulus as described above. In some embodiments, the permission control system 100 could receive data from external systems and monitor 220 the subject based on this external data. For example, in embodiments where the permission control system 100 is associated with a car, the system 100 could receive steering wheel or pedal data from the car, which could be used to indicate lack of attention or erratic behavior. Accordingly, the permission control system 100 could revoke permissions from the subject based on this external data and/or trigger additional rounds of testing, as described above.

In some embodiments, the steps of identifying 204 the individual and providing 208 the stimulus to the individual could be combined together. In such embodiments, the permission control system 100 can provide stimuli and/or tests to the individual that are adapted to both identify the individual (as disclosed in U.S. patent application Ser. No. 17/565,668, for example) and assess the individual for any cognitive impairments.

In some embodiments, the permission control system 100 can further be configured to notify the subject or a third party according to the criteria and a determination as to whether the individual is exhibiting the cognitive impairment. For example, if an individual is seeking to access a building and the permission control system 100 determines that the individual is exhibiting cognitive impairment that could be indicative of a condition (e.g., COVID-19), the system 100 could accordingly notify the security personnel for the building so that they could take any appropriate action.

As noted above, in some embodiments, the permission control system 100 could be incorporated into the various control systems of the location, device, or resource that is being controlled thereby. Further, in other embodiments, the permission control system 100 could be communicably coupled to the various control systems of the location, device, or resource that is being controlled thereby. Accordingly, in various embodiments, the process 200 could be executed by the systems of the location, device, or resource for which access and/or performance of action(s) is being controlled by the permission control system 100. In other embodiments, when the permission control system 100 grants 218 or denies 216 a permission, the permission control system 100 could transmit signals and/or data to the corresponding control system of the location, device, or resource in order to control permission to access to the location, device, or resource accordingly.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values (e.g., variations in the numerical quantity of such values that can occur, but would be recognized to be equivalents by a person skilled in the art).

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

The functions and process steps herein may be performed automatically or wholly or partially in response to user command. An activity (including a step) performed automatically is performed in response to one or more executable instructions or device operations without user direct initiation of the activity.

1. A computer-implemented method for controlling a permission to an environment for an individual by a mobile device, the mobile device comprising a display screen, a detector, and a processor, the method comprising:
presenting, by the display screen, neurophysiological stimuli to the individual;
measuring, by the detector, neurophysiological response by the individual to the neurophysiological stimuli;
determining, by the processor, whether the individual is exhibiting cognitive impairment compatible with COVID-19 based on whether the measured neurophysiological response differs from a reference response by a threshold;
retrieving, by the processor, criteria for the environment from a plurality of criteria, wherein the plurality of criteria are associated with a plurality of different environments;
controlling, by the processor, a permission for the environment according to the criteria and the determination as to whether the individual is exhibiting the cognitive impairment compatible with COVID-19, wherein the permission allows the individual to at least one of access a resource or perform an action associated with the environment;
monitoring, by the processor, the individual as the individual interacts with the resource according to the permission granted and the criteria;
determining, by the processor, whether the individual is exhibiting the cognitive impairment compatible with COVID-19 based on the monitoring; and
revoking, by the processor, the permission in response to the determination that the individual is exhibiting the cognitive impairment compatible with COVID-19.

2. (canceled)

3. The computer-implemented method of claim 1, wherein the permission comprises entering, starting, or driving a motor vehicle.

4. The computer-implemented method of claim 1, wherein the permission comprises accessing a physical or virtual environment.

5. The computer-implemented method of claim 4, wherein the environment comprises a building or other physical structure.

6. The computer-implemented method of claim 1, further comprising:
notifying a third party according to the criteria and the determination as to whether the individual is exhibiting the cognitive impairment compatible with COVID-19.

7. The computer-implemented method of claim 1, further comprising:
identifying the individual based on an adaptive biometric according to the measured neurophysiological response exhibited by the individual.

8. The computer-implemented method of claim 1, wherein the neurophysiological response is measured using at least one of an eye tracking test, a trail-making test, or an eye-hand coordination test.

9. The computer-implemented method of claim 1, wherein the reference response comprises one or more default values.

10. The computer-implemented method of claim 1, wherein the reference response comprises at least one of a characterized response or a baseline response to the neurophysiological stimuli for the individual.

11. The computer-implemented method of claim 1, wherein the reference response comprises at least one of a characterized response of a population of individuals to the neurophysiological stimuli.

12. A system for controlling a permission to an environment for an individual, the system comprising:
a mobile device comprising:
a display screen,
a detector,
a processor, and
a memory coupled to the processor, the memory storing instructions that, when executed by the processor, cause the mobile device to:
present, by the display screen, neurophysiological stimuli to the individual;
measure, by the detector, neurophysiological response by the individual to the neurophysiological stimuli;
determine whether the individual is exhibiting cognitive impairment compatible with COVID-19 based on whether the measured neurophysiological response differs from a reference response by a threshold;
retrieve criteria for the environment from a plurality of criteria, wherein the plurality of criteria are associated with a plurality of different environments;
control a permission for the environment according to criteria and the determination as to whether the individual is exhibiting the cognitive impairment compatible with COVID-19, wherein the permission allows the individual to at least one of access a resource or perform an action associated with the environment,
monitor the individual as the individual interacts with the resource according to the permission and the criteria,
determine whether the individual is exhibiting the cognitive impairment compatible with COVID-19 based on the monitoring, and
revoke the permission in response to the determination that the individual is exhibiting the cognitive impairment compatible with COVID-19.

13. (canceled)

14. The system of claim 12, wherein the permission comprises entering, starting, or driving a motor vehicle.

15. The system of claim 12, wherein the permission comprises accessing a physical or virtual environment.

16. The system of claim 15, wherein the environment comprises a building or other physical structure.

17. The system of claim 12, wherein the memory stores further instructions that, when executed by the processor, cause the mobile device to:
notify a third party according to the criteria and the determination as to whether the individual is exhibiting the cognitive impairment compatible with COVID-19.

18. The system of claim 12, wherein the memory stores further instructions that, when executed by the processor, cause the mobile device to:
identify the individual based on an adaptive biometric according to the measured neurophysiological response exhibited by the individual.

19. The system of claim 12, wherein the neurophysiological response is measured using at least one of an eye tracking test, a trail-making test, or an eye-hand coordination test.

20. The system of claim 12, wherein the reference response comprises one or more default values.

21. The system of claim 12, wherein the reference response comprises at least one of a characterized response or a baseline response to the neurophysiological stimuli for the individual.

22. The system of claim 12, wherein the reference response comprises at least one of a characterized response of a population of individuals to the neurophysiological stimuli.
